SEQUENCE LISTING (2 sequences)

SEQ ID NO: 1 - DNA, 29 bp, Artificial Sequence
  Synthetic Primer
taatgggcccgagtatacggacctttaat

SEQ ID NO: 2 - DNA, 28 bp, Artificial Sequence
  Synthetic Primer
aaatgggcccgattattcataccgtccc

(19) United States
(12) Patent Application Publication
(10) Pub. No.: US 2011/0212526 A1
(43) Pub. Date: Sep. 1, 2011

(54) Method for producing recombinant adeno-associated virus

(75) Inventors: Yu-Chen HU, Hsinchu (TW); Kuo-Shiang HUANG, Hsinchu (TW)

(73) Assignee: National Tsing Hua University, Hsinchu (TW)

(21) Appl. No.: 12/076,379

(22) Filed: Mar. 18, 2008

(51) Int. Cl. C12N 15/00

(52) U.S. Cl. 435/440

(57) ABSTRACT

A method of producing recombinant adeno-associated virus (rAAV) including the following steps of cotransducing host cells with a transduction solution comprising recombinant baculovirus carrying genes of the rAAV, and culturing the cotransduced host cells in a medium.

### BACKGROUND OF THE INVENTION

1. Field of the Invention

The present invention generally relates to a method for producing virus, in particular, to a method of producing recombinant adeno-associated virus.

2. Description of Related Art

Adeno-associated virus (AAV) is a single stranded DNA virus and has been widely utilized as a gene therapy vector. The AAV genome, encompassing the rep and cap genes and the flanking left and right inverted terminal repeats (ITRs), is encapsidated in a non-enveloped icosahedral capsid. The ITRs serve as the primers and origins of replication for DNA replication. The ITRs are also essential for packaging the viral genome into the virus capsid, integration into and excision from host chromosome. The Rep proteins, including Rep78, Rep68, Rep52 and Rep40, are expressed from the endogenous p5 and p19 promoters and the two large Rep proteins are required for replication, regulation of the AAV promoters, site-specific integration and rescue of the AAV genome from its integrated state. The Cap proteins, expressed from the cap gene, include VP1, VP2 and VP3 and are essential for virus assembly. In addition to these gene products, the AAV genome replication requires adenovirus or herpes simplex virus to provide helper functions.

To date, the production of recombinant AAV (rAAV) usually involves the co-transfection of HEK-293 cells with plasmids that carry (1) the vector genome (including the target gene and flanking ITRs), (2) rep and cap genes and (3) helper genes (e.g. adenovirus E2A, E4 and VA RNA). After co-transfection of HEK-293 cells (which stably express adenovirus E1 proteins), E2A genes are driven by E1 and then activate downstream gene expression as well as subsequent genome DNA replication and packaging. In approximately 3 days, the AAV can be harvested from the cells and the medium. Aside from the aforementioned systems, baculovirus is a DNA virus that infects insects and has been widely employed for recombinant protein production in insect cells.

US 2003/0143727 A1 discloses a novel apparatus and method for efficiently cultivating cells with minimal mortality in order to harvest a maximum amount of cellular products generated by the cultivated cells. More particular, this prior art invention teaches a method and a device for plating cells and causing maximum adherence of cells of interest. Furthermore, this prior art invention also teaches a growth substrate means that is capable of providing the largest surface area for cell adhesion and functions as an oxygenator, a depth filter and a static mixer to maximize the production of cellular products by intermittently and periodically provide sufficient oxygen and nutrients to the cells without causing cell death. The device of this prior art invention is economical and can be disposable thus eliminating complications caused by sterilization and is capable of periodically and intermittently provide oxygen and nutrients to cells, through controlling the amount of culture medium that comes into contact with the growth substrate means. The disclosure of US2003/0143727 A1 is incorporated by reference in its entirety.

### SUMMARY OF THE INVENTION

Accordingly, the present invention is directed to methods of producing recombinant adeno-associated virus (rAAV), which have higher efficiencies.

The present invention provides a method of producing recombinant adeno-associated virus (rAAV), comprising the following steps: i) cotransducing host cells with a transduction solution comprising recombinant baculovirus carrying genes of the rAAV; and ii) culturing the cotransduced host cells resulting from step i) in a medium.

Preferably, the transduction solution comprises I) Bac-lacZ, a recombinant baculovirus harboring a reporter gene flanked by adeno-associated virus serotype 2 (AAV-2) inverted terminal repeats (ITRs); and II) Bac-RC, a recombinant baculovirus harboring AAV-2 rep and cap genes. More preferably, Bac-lacZ and Bac-RC in the transduction solution is in a dose ratio of about 1:6.

Preferably, the transduction solution further comprises III) a Bac-Helper, a recombinant baculovirus carrying Ad E2A, E4, and VA RNA genes. More preferably, the transduction solution comprises a multiplicity of infection (MOI) of about 6 of Bac-lacZ, a MOI of about 35 of Bac-RC, and a MOI of about 5 of Bac-Helper.

Preferably, the medium in step ii) comprises 1-10 mM of a butyrate, such as sodium butyrate.

Preferably, the host cells in step i) are immobilized on carriers and the cotransducing in step i) comprises submerging the host cells on the carriers in the transduction solution and exposing the host cells on the carriers to air alternately.

Preferably, the transduction solution is contained in a first chamber, the carriers are contained in a second chamber connected to the first chamber, and the first chamber is compressed and relaxed so as to submerge the host cells on the carriers in the transduction solution and expose the host cells on the carriers to air alternately.

Preferably, the culturing in step ii) comprises submerging the cotransduced host cells on the carriers resulting from step i) in the medium and exposing the cotransduced host cells on carriers to air alternately.

Preferably, the medium is contained in a first chamber, the carriers are contained in a second chamber connected to the first chamber, and the first chamber is compressed and relaxed so as to submerge the host cells on the carriers in the medium and expose the host cells on the carriers to air alternately.

Preferably, the culturing in step ii) further comprises intermittently feeding a fresh medium, which is the same as the medium for culturing the cotransduced host cells, to the cotransduced host cells on the carriers, while maintaining a fixed amount of the medium submerging the cotransduced host cells on the carriers. More preferably, the fresh medium intermittently fed to the cotransduced host cells on the carriers at a rate of two to four times of the amount of the medium for culturing the cotransduced host cells per 24 hours.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention.

FIG. 1A and FIG. 1B are cross-sectional views of a reactor suitable for use in the present invention.

FIG. 2 is a cross-sectional view of another reactor suitable for use in the present invention.

FIG. 3 is a cross-sectional view of the reactor shown in FIG. 2 which is inverted.

FIG. 4 is a cross-sectional view of the reactor shown in FIG. 2 arranged with the system for perfusion operation.

FIG. 5A illustrates the relation between relative virus dosage and rAAV yield measured by Q-PCR in the present invention.

FIG. 5B illustrates the relation between relative virus dosage and rAAV yield measured by titration in the present invention.

FIG. 5C illustrates the relation between concentration of sodium butyrate and rAAV yield measured by Q-PCR in the present invention.

FIG. 5D illustrates the relation between concentration of sodium butyrate and rAAV yield measured by titration in the present invention.

FIG. 6A illustrates the HEK-293 cell growth curves in the present invention.

FIG. 6B illustrates the rAAV yield after transduction measured by qPCR in the present invention.

FIG. 6C illustrates the rAAV yield after transduction measured by titration in the present invention.

### DESCRIPTION OF PREFERRED EMBODIMENTS OF THE INVENTION

The present invention provides a method of producing rAAV. The method includes filling a first chamber of a reactor with a medium. The reactor further includes a second chamber and carriers with host cells immobilized thereon, wherein the carriers are disposed in the second chamber. The first chamber is connected to the second chamber with the carriers on top of the first chamber. After filling the medium, the first chamber is compressed and released so that the carriers can be submerged in the medium and exposed to the air alternately. Then the host cells are transduced with recombinant baculovirus carrying genes of the rAAV. The transduced host cells are then cultured to produce the rAAV. Moreover, the second chamber may include a lid and a neck, and the method may further include seeding the host cells on the carriers by inverting the reactor to settle the carriers and a cell suspension containing host cells on the neck and the lid, and incubating the host cells.

According to an embodiment of the present invention, transducing the host cells includes filling the first chamber of the reactor with transduction solution containing the recombinant baculovirus, compressing the first chamber of the reactor to submerge the carriers in the transduction solution, releasing the first chamber of the reactor to expose the carriers to air, and repeating the steps of compressing and releasing the first chamber, so that the carriers can be submerged in the cell suspension and exposed to the air alternately.

According to another embodiment of the present invention, culturing the transduced host cells includes filling the first chamber of the reactor with a medium, compressing the first chamber of the reactor to submerge the carriers in the medium, releasing the first chamber of the reactor to expose the carriers to air, and repeating the steps of compressing and releasing the first chamber, so that the carriers can be submerged in the medium and exposed to the air alternately.

As described above, in the present invention, recombinant baculovirus is employed to mediate the transduction of rAAV genes into the host cells hence boosts the efficiency of production. Moreover, the compression of the first chamber can submerge the carriers in the transduction solution, or medium, for virus transduction, or nutrient transfer, respectively; the relaxation of the first chamber can expose the carriers to the air to allow oxygen transfer. Thus, alternately compressing and releasing the first chamber can put the carriers under different environment alternately, therefore further raises the productivity of the method.

Reference will now be made in detail to the embodiments of the present invention, examples of which are illustrated in the accompanying drawings. Wherever possible, the same reference numbers are used in the drawings and the description to refer to the same or like parts.

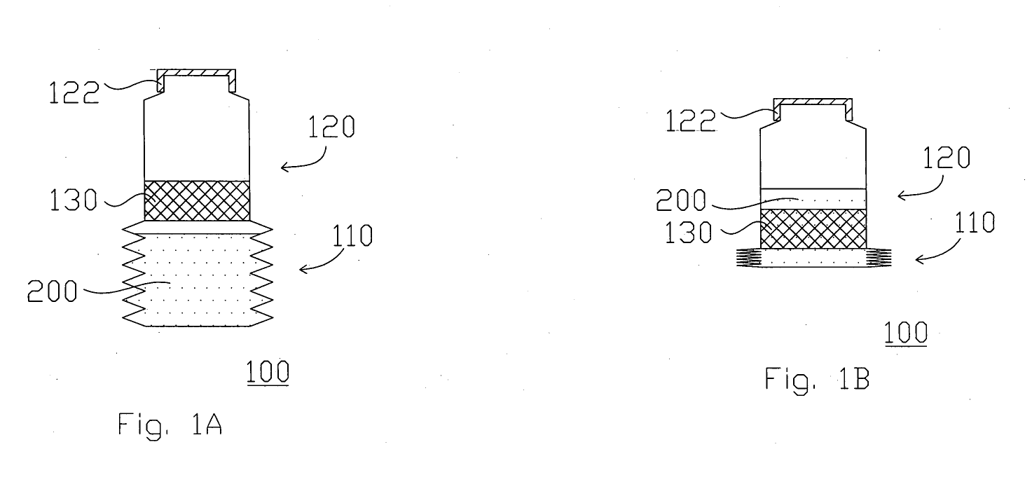

FIG. 1A and FIG. 1B are cross-sectional views of a reactor used in an embodiment of the present invention. A suitable example of the reactor shown in FIGS. 1A and 1B is available from CESCO bioengineering Inc. (Hsinchu, Taiwan) under the code name of BelloCell-500. The reactor 100 includes a first chamber 110, a second chamber 120, and a plurality of carriers 130. The first chamber 110 is connected with the second chamber 120, and the carriers 130 are disposed in the second chamber 120. In the present embodiment, the second chamber 120 has a lid 122 with a filter (not shown in the figures) for ventilation. Moreover, the second chamber 120 has an upper support screen and a lower support screen (not shown in the figures) to confine carriers 130, for example BioNOC II carriers available from CESCO bioengineering Inc. (Hsinchu, Taiwan).

Cotransducing host cells immobilized on the carriers 130 with a transduction solution 200 comprising recombinant baculovirus carrying genes of the rAAV is carried out by introducing the transduction solution into the first chamber 110. The first chamber 110 is then compressed and released alternately. The compression raises the transduction solution 200 to submerge the carriers 130, as shown in FIG. 1B. After a period of delay time at the top, the relaxation drops the transduction solution 200 to the first chamber 110, thus exposing the carriers 130 to air for oxygen transfer. After another delay at the bottom, the cycle is repeated.

Afterwards, the transduced host cells are cultured for rAAV production. In the present embodiment, culturing the transduced host cells is performed by continuing to operate the reactor 100 in the manner described above, except the transduction solution 200 in the first chamber 110 is replaced with a medium. After a period of the culturing, the used medium may be replaced with a fresh medium when needed.

Since recombinant baculovirus is employed to mediate the transduction of rAAV genes into the host cells, thus raises the efficiency of the transduction. Besides, compressing and releasing the first chamber 110 can alternately submerge the carriers 130 in the transduction solution 200 or the medium for transducing or culturing, and expose the carriers 130 to the air to allow oxygen transfer. Combined this with the gene transfer with recombinant baculovirus, the productivity of rAAV of the method can be further boosted.

In FIG. 1A and FIG. 1B, the working volume of the reactor 100 is 500 ml, and the second chamber 120 has a lid 122 equipped with a 0.22 μm filter for ventilation.

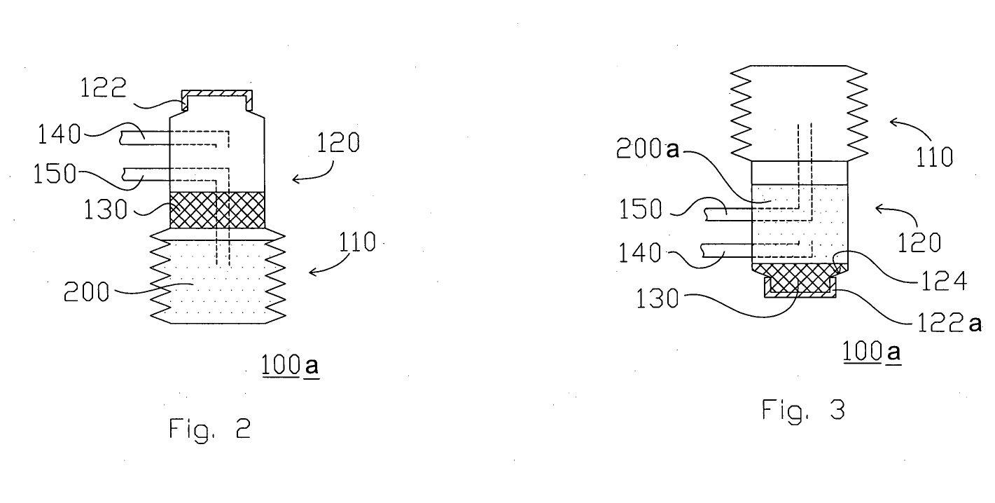

FIG. 2 is a cross-sectional view of another reactor suitable for used in another embodiment according to the present invention, for example the BelloCell-500AP reactor available from CESCO bioengineering Inc. (Hsinchu, Taiwan). This reactor 100a is similar to the reactor 100 shown in FIGS. 1A and 1B, wherein like numerals refer to like elements or parts. The differences are the former (the reactor 100a) additionally has an inlet tube 140 and an outlet tube 150 for perfusion operation and has no upper support screen to confine the carriers 130. As shown in FIG. 2, one end of the inlet tube 140 is connected to and inserted into the second chamber 120, and one end of the outlet tube 150 is connected to the second chamber 120 and extends down to the upper half of the first chamber 110.

FIG. 3 is a cross-sectional view of the reactor 100a shown in FIG. 2, except that the reactor 100a in FIG. 3 is inverted and the lid 122a in FIG. 3 is a normal lid without a filter. Since the reactors 100a has no upper support screen to confine the carriers 130 and the lid 122a is a normal lid without a filter, a cell suspension 200a introduced into the first chamber 110 and the carriers 130 will be settled on the neck 124 the lid 122a when the reactor 100a is inverted, as shown in FIG. 3. Thereby, cell seeding is carried out in the reactor 100a shown in FIG. 3 to immobilize host cells contained in the cell suspension 200a onto the carriers 130. Upon completion of the cell seeding, the reactor 100a is inverted, replenished with a fresh medium, and replaced with a new lid 122 (with a filter) as shown in FIG. 2. Culturing the host cells, cotransducing the host cells, and culturing the cotransduced host cells are then carried out in the reactor 100a similar to the embodiment using the reactor 100 described above, except that an additional perfusion operation is performed throughout the culture periods in the reactor 100a.

The host cells are co-transduced with several recombinant baculoviruses. The reactor 100a is filled with a transduction solution 200, which includes medium and the recombinant baculoviruses. Then, the first chamber 110 is compressed and released alternately, in the manner mentioned in the previous embodiments. The compression raises the transduction solution to submerge the carriers 130. After a period of delay time at the top, the relaxation drops the transduction solution to the first chamber 110, thus exposing the carriers 130 to air for oxygen transfer. After another delay at the bottom, the cycle is repeated.

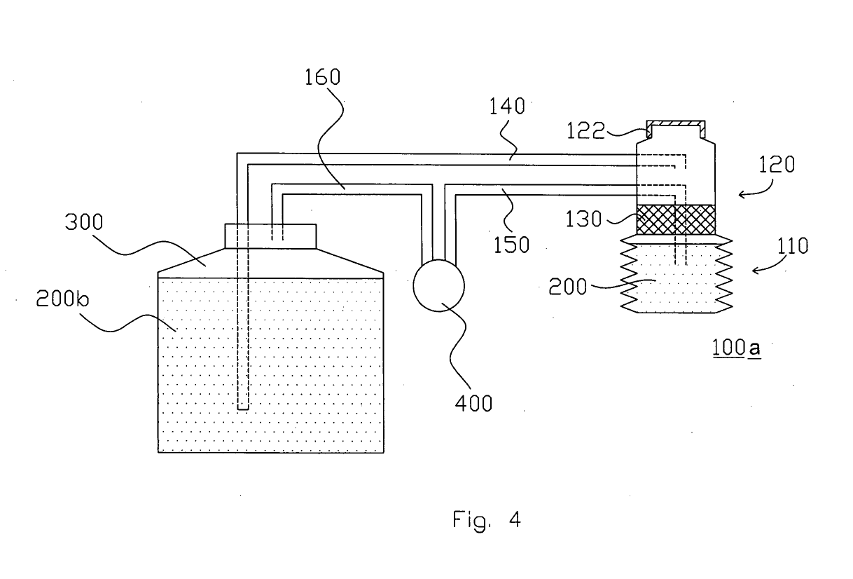

In the present embodiment, the steps of culturing the host cells and culturing the transduced host cells include compressing and releasing the first chamber 110 alternately. The compression raises the medium to submerge the carriers 130. After a period of delay time at the top, the relaxation drops the medium to the first chamber 110, thus exposing the carriers to air for oxygen transfer. Throughout the culture periods, a perfusion operation is performed. The perfusion operation may be implemented as in the following. Referring to FIG. 4, another end of the inlet tube 140 is connected to a 2000-ml external medium reservoir 300 containing a medium 200b and below the medium 200b level, and another end of the outlet tube 150 is connected to a peristaltic pump 400. The peristaltic pump 400 is connected to a confined space (smaller than 500 ml) above the medium 200b level in the external medium reservoir 300 via another tube 160. The pump 400 is turned on and operated intermittently to withdraw a fixed amount of the medium 200 from the first chamber 110 to the reservoir 300, and at the same time an equivalent amount of a fresh medium 200b is perfused from the reservoir 300 through the inlet tube 140 to the carriers 130.

Without intending to limit it in any manner, the present embodiment will be further illustrated by the following example using the present embodiment.

The recombinant baculovirus used in the present examples can be prepared as following. Insect cells (Sf-9) for baculovirus generation and propagation were cultured in TNM-FH medium containing 10% fetal bovine serum (FBS). HEK-293 and HT-1080 cells were cultured in Dulbecco's modified Eagle medium (DMEM) containing 10% FBS. Construction of recombinant baculoviruses started with deletion of the polyhedrin promoter in the donor plasmid pFastBac Dual. To the end, the polymerase chain reaction (PCR) was performed with pFastBac Dual as the template with two synthetic primers (5′-TAATGGGCCCGAGTATACGGACCTTTAAT-3′ and 5′-AAATGGGCCCGATTATTCATACCGTCCC-3′). The amplicon (5.0 kb) was treated with Apa I and ligated to form the plasmid pFastBacΔpolh, which was identical to pFastBac Dual except that the polyhedrin promoter upstream of multiple cloning site I (MCS I) was removed. The recombinant donor plasmids, pBac-LacZ, pBac-RC, and pBac-Helper, were constructed by separately subcloning the genes in pAAV-LacZ, pAAV-RC, and pHelper plasmids into pFastBacΔpolh.

Specifically, pAAV-LacZ contained the cytomegalovirus (CMV) promoter-driven lacZ flanked by AAV-2 left and right ITRs. The complete 4.7 kb cassette was digested with PstI, inserted into pBluescript II KS+, and then subcloned into MSC I of pFastBacΔpolh by treatment with PstI and HindIII. The recombinant plasmid was designated pBac-LacZ. To construct pBac-RC, the AAV-2 rep and cap genes (≈4.3 kb) harbored by pAAV-RC were cleaved by EcoRV and SmaI and subcloned into MCS I of pFastBacΔpolh by StuI treatment. pBac-Helper was constructed in two stages. The gene fragment encoding nucleotides 1-1692 of the adenovirus E2A gene was cleaved from pHelper with KpnI and XhoI and subcloned into MCS I of pFastBacΔpolh to form pBac-E2A. The gene fragment encompassing the rest of the E2A gene and the downstream E4 and VA RNA genes carried by pHelper was then cleaved with XhoI and SalI, treated with calf intestine alkaline phosphatase, and subcloned into pBac-E2A downstream of E2A. The resultant plasmid harboring the adenovirus E2A/E4/VA RNA genes (9.3 kb) was designated pBac-Helper. The recombinant baculoviruses Bac-LacZ, Bac-RC and Bac-Helper were generated. The baculoviruses were passaged by infecting insect cells at a multiplicity of infection (MOI) of 0.1, harvested 4 days postinfection, and treated by the end-point dilution method. The viruses were not concentrated by ultracentrifugation.

For cell seeding, 1×10⁸ HEK-293 (Human Embryonic Kidney) cells suspended in 150 ml DMEM were added to the reactor 100a shown in FIG. 2. The reactor 100a was inverted (FIG. 3), swirled gently so that the cells and carriers 130 were evenly settled to the neck 124 and lid 122a (normal lid without the 0.22 μm filter) of the reactor 100a, and incubated in the CO₂ incubator at 37° C. After 3 h, the reactor 100a was inverted back to normal position, replenished with 350 ml fresh medium, and replaced with a new lid 122 (with a 0.22 μm filter) (FIG. 2).

The linear moving rate was set at 1.5 mm/s (delay time at the top and bottom is 0 s and 30 s, respectively) throughout the culture period. One milliliter medium was withdrawn daily from the sampling port and measured for glucose concentration. The reactor 100a in the present example was equipped with the inlet tube 140 and the outlet tube 150 for perfusion operation. The inlet tube 140 and the outlet tube 150 were connected to a 2-liter external medium reservoir and a peristaltic pump as described above. At 24 h post-seeding, the pump was turned on and operated intermittently at a perfusion rate of 999 ml medium per 24 h. The perfusion rate was elevated to 1999 ml medium per 24 h after the cells entered exponential growth phase.

Because the Rep expression level was crucial to rAAV yield and baculovirus dosage profoundly influences the protein expression level, we sought to manipulate the baculovirus dosage to improve the rAAV yield. To this end, HEK-293 cells were co-transduced in the 10-cm dishes (5×10⁶ cells/dish) using varying relative dosages of Bac-LacZ and Bac-RC. To simplify the experiment design, the Bac-Helper dosage (MOI=5) and total baculovirus dosage (MOI=45) were fixed. HEK-293 cells plated onto 10-cm dishes (5×10⁶ cells/dish) overnight were incubated with unconcentrated virus using phosphate-buffered saline (PBS, pH 7.4) as the surrounding solution to adjust the final volume to 3 ml. The virus solution volume varied depending on the MOI used. The dishes were shaken on the rocking plate for 6 h at 27° C. After transduction, the cells were washed with PBS, replenished with 10 ml DMEM and continued to be cultured at 37° C.

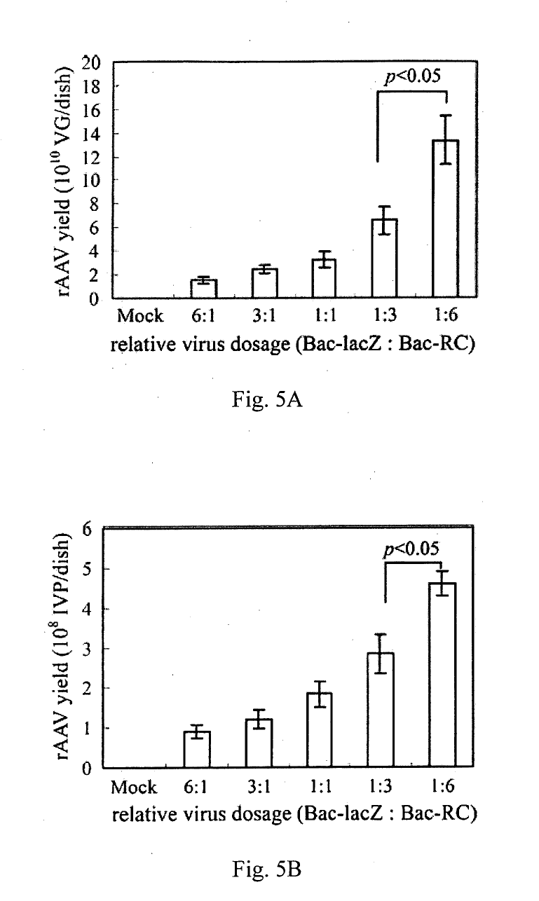

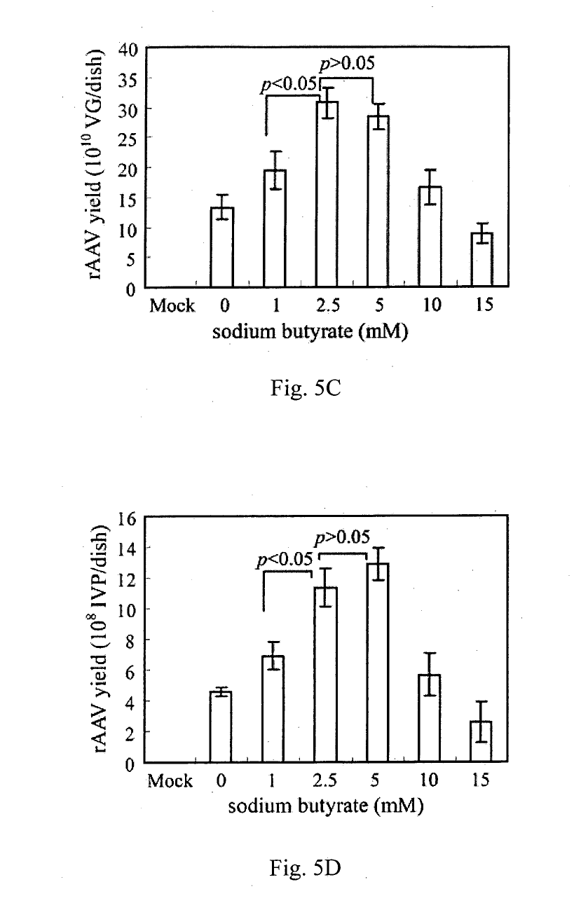

Referring to FIG. 5A and FIG. 5B, both the Q-PCR (FIG. 5A) and titration (FIG. 5B) data measured at 3 days post-transduction (dpt) depict that the rAAV yield is low at low Bac-RC dosages (e.g. dosage ratio of Bac-LacZ:Bac-RC at 6:1), but increases upon elevated Bac-RC dosage. In comparison with the dosage ratio of 1:1, the maximum rAAV yield resulting from the dosage ratio of 1:6 was ≈4.2-fold higher in vector genome (≈1.3×10¹¹ VG/dish) and 2.6-fold higher in titer (≈4.6×10⁸ IVP/dish or 92 IVP/cell). These data underscore the significance of the relative expression levels and indicate that a higher dosage of Bac-RC relative to Bac-LacZ favors the rAAV production.

It is also noteworthy that manipulation of the baculovirus dosage may improve the rAAV yield. Referring to FIG. 5C and FIG. 5D, to further elevate the rAAV yield, HEK-293 cells were co-transduced using the best conditions identified above (MOI ratio of Bac-LacZ:Bac-RC=1:6, total MOI=45) and then cultured with DMEM containing various concentrations of sodium butyrate, a histone deacetylase inhibitor known to enhance baculovirus-mediated gene expression in mammalian cells. The qPCR (FIG. 5C) and titration (FIG. 5D) data both reveal that the rAAV yield increases with ascending butyrate concentrations, reaching a plateau and then declines, probably due to the elevated cytotoxicity imparted by butyrate. In comparison with 0 mM butyrate, 2.5 mM results in a ≈1.3-fold increase in vector genome number (≈3.1×10¹¹ VG/dish, ≈6.2×10³ VG/cell) while 5 mM give rise to a ≈2.0-fold increase in biologically active particles (≈1.4×10⁹ IVP/dish or ≈280 IVP/cell). Since the rAAV yields obtained at 2.5 and 5 mM are statistically similar (p>0.05), 2.5 mM butyrate can be applied in the embodiments of the present invention.

HEK-293 cells cultured in the reactor 100a were co-transduced with Bac-LacZ, Bac-RC and Bac-Helper. The cell number was counted and the three viruses for co-transduction were mixed. The volume of each virus depended on the MOI to be used. The transduction solution was prepared by mixing 167 ml virus solution with 333 ml NaHCO₃-deficient DMEM (serving as the surrounding solution to adjust the final volume to 500 ml), which gave a volumetric ratio (surrounding solution to virus solution) of ≈2 that favored the virus-like particle production. The pH of the transduction solution was adjusted to 7.7 with 1 N NaOH.

Prior to transduction, the spent medium was poured and the immobilized cells were washed with 400 ml PBS for 5 min. After washing, PBS was discarded and the transduction was initiated by adding the 500 ml transduction solution. The reactor was swirled several times to allow uniform contact between cells and viruses, and the transduction continued for 6 h by operating the reactor 100a (linear moving rate was 1.5 mm/s, delay time at the top and bottom was 20 s). Then the transduction solution was poured and 470 ml fresh medium containing 2.5 mM sodium butyrate was added.

The rAAV production phase was commenced by operating the reactor 100a at 37° C. (linear moving rate is 1.5 mm/s, delay time at the top and bottom is 0 and 30 s, respectively). The production was terminated 4 days post-transduction (dpt).

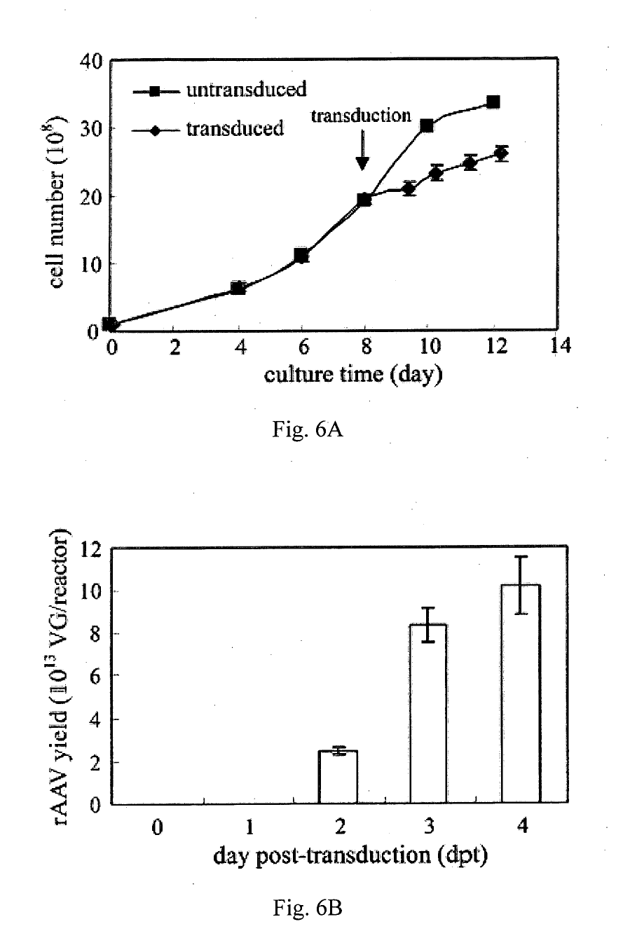

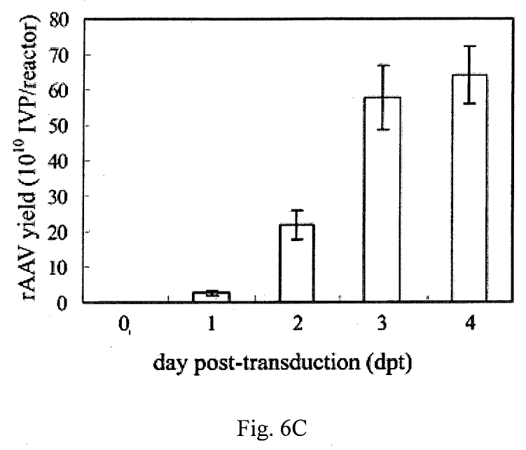

HEK-293 cells were either untransduced or co-transduced with Bac-LacZ (MOI=6), Bac-RC (MOI=35) and Bac-Helper (MOI=5) when the cell number reached 2.0x·10⁹, and then cultured with DMEM containing 2.5 mM butyrate. As shown in FIG. 6A, the transduced cells remain capable of growth in the reactor 100a, although at a slower rate, which is ascribed to the shift of metabolic activities from cell growth to protein over-expression. It is noteworthy that during transduction NaHCO₃-deficient DMEM was as the surrounding solution in lieu of PBS because PBS caused serious detachment of HEK-293 cells. Replacement of PBS with NaHCO₃-deficient DMEM considerably alleviates the cell detachment problem while maintain the high transduction efficiency. During the rAAV production phase, approximately 1×10⁸ cells were collected from the reactor daily and the samples were purified by CsCl gradient ultracentrifugation. The rAAV yield was quantified by Q-PCR (FIG. 6B) and virus titration (FIG. 6C). FIGS. 6B and 6C confirm that the yield increases with time but the production rate slows by 3 days post-transduction as the yield on days 3 and 4 is statistically similar (p>0.05). For each reactor run, the maximum yield at 4 days post-transduction reaches ≈3.8×10⁴ VG/cell and ≈247 IVP/cell, respectively. These yields correspond to ≈1×10¹⁴ VG (FIG. 6B) and ≈6.4×10¹¹ IVP (FIG. 6C) per reactor run.

In summary, in the present invention, recombinant baculovirus are utilized to mediated the transduction of the host cells, which raises the efficiency of the method. Moreover, compression and relaxation of the first chamber can be employed in different stages of the method, such as cell culturing, and cell transduction. The compression and relaxation of the first chamber can put the carriers under different environments, thus boosts the productivity of the method.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims and their equivalents.

1. A method of producing recombinant adeno-associated virus (rAAV), comprising the following steps:
i) cotransducing host cells with a transduction solution comprising the following recombinant baculoviruses: Bac-lacZ, Bac-RC and Bac-Helper; and
ii) culturing the cotransduced host cells resulting from step i) in a medium,
wherein said host cells are mammalian cells; said Bac-lacZ is a recombinant baculovirus harboring a reporter gene flanked by adeno-associated virus serotype 2 (AAV-2) inverted terminal repeats (ITRs); said Bac-RC is a recombinant baculovirus harboring AAV-2 rep and cap genes; and said Bac-Helper is a recombinant baculovirus harboring adenovirus E2A, E4, and VA RNA genes,
wherein the host cells in step i) are immobilized on carriers and the cotransducing in step i) comprises submerging the host cells on the carriers in the transduction solution and exposing the host cells on the carriers to air alternately,
wherein the culturing in step ii) comprises submerging the cotransduced host cells on the carriers resulting from step i) in the medium and exposing the cotransduced host cells on carriers to air alternately,
wherein the medium is contained in a first chamber, the carriers are contained in a second chamber connected to the first chamber, and the first chamber is compressed and relaxed so as to submerge the cotransduced host cells on the carriers in the medium and expose the host cells on the carriers to air alternately, and
wherein the culturing in step ii) further comprises a perfusion operation which is performed throughout the culturing period, said perfusion operation comprising intermittently feeding a fresh medium, which is the same as the medium for culturing the cotransduced host cells, to the cotransduced host cells on the carriers, and intermittently withdrawing the medium from the first chamber in an amount equivalent to the feeding amount of the fresh medium, so that a fixed amount of the medium submerging the cotransduced host cells on the carriers is maintained.

2. (canceled)

3. The method according to claim 1, wherein Bac-lacZ and Bac-RC in the transduction solution is in a dose ratio of about 1: 6.

4. (canceled)

5. The method according to claim 1, wherein the transduction solution comprises a multiplicity of infection (MOI) of about 6 of Bac-lacZ, a MOI of about 35 of Bac-RC, and a MOI of about 5 of Bac-Helper.

6. The method according to claim 1, wherein the medium in step ii) comprises 1-10 mM of a butyrate.

7. The method according to claim 6, wherein the butyrate is sodium butyrate.

8. The method according to claim 1, wherein the host cells in step i) are immobilized on carriers and the cotransducing in step i) comprises submerging the host cells on the carriers in the transduction solution and exposing the host cells on the carriers to air alternately.

9. The method according to claim 8, wherein the transduction solution is contained in a first chamber, the carriers are contained in a second chamber connected to the first chamber, and the first chamber is compressed and relaxed so as to submerge the host cells on the carriers in the transduction solution and expose the host cells on the carriers to air alternately.

10. The method according to claim 8, wherein the culturing in step ii) comprises submerging the cotransduced host cells on the carriers resulting from step i) in the medium and exposing the cotransduced host cells on carriers to air alternately.

11. The method according to claim 10, wherein the medium is contained in a first chamber, the carriers are contained in a second chamber connected to the first chamber, and the first chamber is compressed and relaxed so as to submerge the host cells on the carriers in the medium and expose the host cells on the carriers to air alternately.

12. The method according to claim 10, wherein the culturing in step ii) further comprises intermittently feeding a fresh medium, which is the same as the medium for culturing the cotransduced host cells, to the cotransduced host cells on the carriers, while maintaining a fixed amount of the medium submerging the cotransduced host cells on the carriers.

13. The method according to claim 12, wherein the fresh medium is intermittently fed to the cotransduced host cells on the carriers at a rate of two to four times of the amount of the medium for culturing the cotransduced host cells per 24 hours.

14. The method according to claim 1, wherein said mammalian cells are Human Embryonic Kidney (HEK)—293 cells.

15. The method according to claim 10, wherein said mammalian cells are HEK-293 cells.

16. The method according to claim 11, wherein said mammalian cells are HEK-293 cells.

17. The method according to claim 12, wherein said mammalian cells are HEK-293 cells.

18. The method according to claim 13, wherein said mammalian cells are HEK-293 cells.
